(19) United States
(12) Patent Application Publication
(10) Pub. No.: US 2025/0331810 A1
(43) Pub. Date: Oct. 30, 2025

(54) IMAGE-BASED ESTIMATION OF LEFT VENTRICULAR MYOCARDIAL STIFFNESS

(71) Applicant: UNIVERSITY OF SOUTH CAROLINA, COLUMBIA, SC (US)

(72) Inventors: FRANCIS SPINALE, BLYTHEWOOD, SC (US); TAREK SHAZLY, COLUMBIA, SC (US)

(21) Appl. No.: 19/064,891

(22) Filed: Feb. 27, 2025

Related U.S. Application Data
(60) Provisional application No. 63/639,731, filed on Apr. 29, 2024.
(60) Provisional application No. 63/666,840, filed on Jul. 2, 2024.

(51) Int. Cl. A61B 8/08; A61B 8/00

(52) U.S. Cl. CPC A61B 8/0883; A61B 8/485; A61B 8/5223

(57) ABSTRACT

The disclosure deals with methodology and corresponding apparatus/system subject matter for image-based estimation of left ventricular myocardial stiffness. Increased left ventricular myocardial stiffness is a key factor in the development and progression of heart failure. Despite the potential impact on the clinical management of heart failure, there is currently a lack of available techniques to assess left ventricular myocardial stiffness. To address this limitation, a simple protocol is disclosed for processing routine echocardiographic imaging data to estimate left ventricular myocardial stiffness, with protocol specification for patients at risk for heart failure with preserved ejection fraction, for both sensitivity and translational feasibility of the obtained estimates of left ventricular myocardial stiffness.

### PRIORITY CLAIM

The present application claims the benefit of priority of U.S. Provisional Patent Application No. 63/639,731, filed Apr. 29, 2024, and the benefit of priority of U.S. Provisional Patent Application No. 63/666,840, filed Jul. 2, 2024, both of which are titled Image-Based Estimation Of Left Ventricular Myocardial Stiffness, and both of which are fully incorporated herein by reference for all purposes.

### STATEMENT REGARDING FEDERALLY SPONSORED RESEARCH OR DEVELOPMENT

This invention was made with government support under Grant No. 1R01HL130972-01A1, awarded by the National Institutes of Health. The government has certain rights in the invention.

### BACKGROUND OF PRESENTLY DISCLOSED SUBJECT MATTER

The disclosure deals with methodology and corresponding apparatus/system subject matter for image-based estimation of left ventricular myocardial stiffness. In the field of cardiac-related subject matter, the presently disclosed subject matter variously relates to concept such as left ventricle structure, heart failure, myocardial stiffness, speckle tracking echocardiography, and ultrasound-based sensing.

### 1. Introduction

Left ventricle (LV) remodeling, which alters both the geometry and mechanical properties of the myocardium, is a deterministic process in the development and progression of heart failure (HF). As such, sensitive techniques to track the rate and extent of LV remodeling are necessary to evaluate patient risk and guide treatment protocols. Echocardiography has become the gold standard for assessing the structure and function of the heart. Moreover, recent advancements in both hardware and software have given rise to a relatively new echocardiographic capability: the assessment of regional LV myocardial strain through two-dimensional speckle tracking echocardiography (STE). While this capability has been shown to be clinically useful in a variety of disease states, the fundamental dependency of strain on hemodynamic load and LV geometry has diminished its widespread clinical utility and render it inept to detect certain modes of HF in which an increase in LV myocardial stiffness (despite relatively preserved strain) plays a key role in disease progression.

Heart failure (HF) continues to be a leading cause of death and disability, and despite advances in HF management, the HF burden continues to rise at an alarming rate (Heidenreich et al., 2022; Redfield and Borlaug, 2023). Using available Medicare data, hospital readmission due to HF have estimated costs exceeding $50 billion by next year and approaching $100 billion by 2030—costs that are certain to impact medical resources and reach an unsustainable level (Jin et al., 2022; Mohebi et al., 2022).

One major barrier to preventing HF progression is that while patients may present with clinical symptoms of HF, the underlying pathophysiology can be quite different. One ever increasing and particularly challenging pathophysiological classification is HF with preserved ejection fraction (HFpEF) (Behnoush et al., 2023; Li et al., 2022; Omote et al., 2022). Unlike HF with reduced ejection fraction (HFrEF), wherein compromised left ventricular (LV) pump function can be easily detected, HFpEF has a subtle and insidious progression because conventional imaging approaches such as ultrasound (echocardiography) are not able to identify its early development (Obokata et al., 2020). The underlying pathophysiology of HFpEF is that of primarily diastolic dysfunction, specifically due to increases in LV wall thickness and LV myocardial stiffness that together cause an increase in LV chamber stiffness. Increased LV chamber stiffness in turn leads to elevated LV filling pressures and ultimately the development of HF signs and symptoms. Currently, echocardiographic methods cannot directly measure a key response variable in HFpEF development and progression, namely LV myocardial stiffness, and thus this critical functional milestone is not available for clinical decision making.

The present disclosure addresses this unmet medical need by developing algorithms which take advantage of speckle tracking echocardiography (STE) to establish a straightforward approach to estimate LV myocardial stiffness, where the requisite data processing can be readily integrated into a conventional echocardiography workflow/workstation.

STE provides a non-invasive tool to quantify regional LV myocardial strain based on relative changes in a segment length, whereby segments are defined with respect to acoustic markers (speckles) that are contained within a prespecified myocardial region and tracked over the cardiac cycle (Bansal and Kasliwal, 2013). In the LV short-axis echocardiographic view, regional LV myocardial strain/strain rate can be obtained in the radial and circumferential directions using correspondingly oriented segments; in the LV long-axis view, regional radial and longitudinal LV myocardial strain/strain rate can be similarly measured. Thus, STE-based strain measures are linearized approximations of the finite normal strain components that would, in addition to shear strain components, be appropriate for a complete description of LV myocardial deformation in the framework of continuum solid mechanics (Collier et al, 2017). While these approximations limit the applicability of STE-based LV myocardial strain in fundamental mechanics, they are clinically-accessible and have demonstrated utility for HF (primarily HFrEF) diagnosis and prognosis in both pre-clinical and clinical settings (Chan et al., 2006; Gjesdal et al., 2008; Haugaa et al., 2013; Reant et al., 2008; Stanton et al., 2009; Sun et al., 2007; Torres et al., 2018).

STE-based LV myocardial strains depend on LV mechanical loading and geometry, as well as the mechanical properties of the LV myocardium. The LV myocardium exhibits layer-specific passive and active mechanical properties, with complex constitutive behavior that includes significant nonlinearity and anisotropy, undergoes both normal and shear strains under physiological loading, and contains residual strains (Liu and Wang, 2019; Mehrotra et al., 2022; Omens and Fung, 1990). Therefore, consideration of STE-based LV myocardial strain alone is inadequate for constitutive modeling of the LV myocardium, and perhaps as a result, indices of LV myocardial mechanical properties are not routinely considered in a clinical setting. We posit that the lack of translational methods to estimate LV myocardial mechanical properties represents a critical knowledge gap, as an increase in LV myocardial stiffness due to maladaptive LV remodeling underlies various modes of HF (Grossman et al., 1974; Jalil et al., 1989; Torres et al., 2018; Torres et al., 2020; Yarbrough et al. 2012) and provides a direct or indirect therapeutic target for diverse cardiac interventions (Sakata et al., 2013). We seek to address this knowledge gap with a simple protocol to estimate LV myocardial mechanical properties via coupling of STE-based LV myocardial strain measures and approximations of regional LV myocardial stress. The presently disclosed protocol is based on simple processing of readily-available clinical data, and thus could be easily incorporated into current software for STE analysis and rapidly translated to a clinical translation.

### SUMMARY OF PRESENTLY DISCLOSED SUBJECT MATTER

The disclosure deals with methodology and corresponding apparatus/system subject matter for image-based estimation of left ventricular myocardial stiffness. Increased left ventricular myocardial stiffness is a key factor in the development and progression of heart failure. Despite the potential impact on the clinical management of heart failure, there is currently a lack of available techniques to assess left ventricular myocardial stiffness. To address this limitation, a simple protocol is disclosed for processing routine echocardiographic imaging data to estimate left ventricular myocardial stiffness, with protocol specification for patients at risk for heart failure with preserved ejection fraction, for both sensitivity and translational feasibility of the obtained estimates of left ventricular myocardial stiffness.

The presently disclosed subject matter is based on a novel and non-obvious extension of the two-dimensional STE that allows for estimation of LV myocardial stiffness, therefore providing a metric with direct relevance to HF assessment. We have developed the presently disclosed approach in preclinical models of HF, with an algorithm that integrates STE-based LV myocardial strain measures and approximations of LV myocardial stress in a manner that could be easily incorporated into current software for STE analysis and rapidly translated to a clinical translation.

Sensitive techniques to track the rate and extent of LV remodeling are necessary to evaluate risk and treatment options on a patient-specific basis. A novel extension to speckle-tracking echocardiography technology has been developed as a means to non-invasively identify the mechanical properties of the LV myocardium. This presently disclosed technology can be implemented as a post-processing complement to traditional echocardiographic studies to provide a detailed biomechanical analysis of the changing heart as it pertains to disease progression, without the need for complex analysis/modeling.

The presently disclosed subject matter/technology can be used as a complement to standard echocardiographic analysis as a sensitive biomechanical marker of the rate and extent of LV remodeling. This analysis can be completed as a post-processing step from images that are routinely acquired in a complete transthoracic echocardiographic study based on only algebraic calculations and specified to distinct myocardial regions.

Additional applications: While the disclosure presented herein specifically relates to the LV of the heart, the same methodology can be extended to other soft tissues within the body. This extension would be contingent on two factors: (1) access to an image modality which would allow for the successful tracking of the tissue deformation in response to a given load and (2) an accurate estimation of the in-vivo load exerted on the tissue. As an example, thoracic or abdominal aortic aneurysms would be an ideal application for this technology. First, the irregular geometries of these structures negate the use of analytical approaches to identify constitutive model parameters and/or estimate the wall stress distribution. Furthermore, STE and magnetic resonance have been previously applied to the aorta to track the deformation of the aorta during and after ventricular ejection. Finally, doppler echocardiography can be used to generate a reasonable estimation of pressure. Given the fact that surgical repair of aortic aneurysms carries a mortality rate approaching 10%, this detailed mechanical analysis would provide surgeons with complementary data to inform their decision on whether-or-not to surgically intervene.

At present writing, no other marketplace product is known to us which utilizes myocardial strain imaging in an inverse framework to identify mechanical properties of the heart. The diagnostic imaging and monitoring global market is around $25 billion, and about $10 billion in the United States. In the US, there are currently 5.7 million people diagnosed with heart failure and these patients receive an echocardiographic examination on average 1.3 times per year. There are currently no products, services, or processes that can provide this type of mechanical analysis. Ultimately, the stiffness indices we can generate will provide information about the regional mechanical properties of the LV myocardium when under either compression or tension. This presently disclosed technology will provide clinicians with a diagnostic advantage as they look to assess the progression of heart disease and/or the effectiveness of treatment strategies.

It is to be understood that the presently disclosed subject matter equally relates to apparatus or systems as well as associated and/or corresponding methodologies. One exemplary such methodology relates to methodology for using echocardiography imaging to evaluate patient heart failure risk for guiding treatment protocols, comprising acquiring and processing echocardiographic imaging data for the left ventricle (LV) of a patient's heart; using the imaging data to calculate regional LV myocardial strains; estimating mean regional LV wall stress by calculating the mean LV myocardial stresses associated with the calculated regional LV myocardial strains; using the calculated regional LV myocardial strains and associated LV myocardial stresses to estimate regional LV myocardial stiffness; and using the estimated regional LV myocardial stiffness to gauge development and progression of patient heart failure.

Another exemplary such method comprises methodology for using short-axis left ventricle (LV) echocardiograms for estimating LV myocardial stiffness echocardiography imaging to evaluate patient heart failure risk for guiding treatment protocols, comprising acquiring and processing short-axis LV echocardiographic imaging data for the LV at the level of the papillary muscles of respective targets of a patient's heart; calculating regional LV myocardial strains (ε) includes imaging mid-wall LV myocardial deformation from end-systole (ES) to the end-diastole (ED); estimating mean regional LV wall stress (σ) by calculating the mean LV myocardial stresses associated with the calculated regional LV myocardial strains; estimating regional LV myocardial stiffness (KM) by using the slope of the linearized regional stress/strain relation from the calculated regional LV myocardial strains and associated LV myocardial stresses; and using the estimated regional LV myocardial stiffness to gauge development and progression of patient heart failure.

Yet another exemplary presently disclosed methodology relates to methodology for using imaging to estimate soft tissue conditions within the body for evaluating patient risk and guiding treatment protocols, comprising acquiring image data of a target soft tissue location within a patient's body while the location is subjected to a given load; estimating the in-vivo load exerted on the tissue; using the image data for tracking the deformation of the soft tissue location when subjected to the given load, to determine mechanical properties of the target soft tissue location; and using the determined mechanical properties to evaluate patient risk for guiding treatment protocols.

Other example aspects of the present disclosure are directed to systems, apparatus, tangible, non-transitory computer-readable media, user interfaces, memory devices, and electronic devices for image-based estimation of left ventricular myocardial stiffness technology. To implement methodology and technology herewith, one or more processors may be provided, programmed to perform the steps and functions as called for by the presently disclosed subject matter, as will be understood by those of ordinary skill in the art.

Additional objects and advantages of the presently disclosed subject matter are set forth in, or will be apparent to, those of ordinary skill in the art from the detailed description herein. Also, it should be further appreciated that modifications and variations to the specifically illustrated, referred and discussed features, elements, and steps hereof may be practiced in various embodiments, uses, and practices of the presently disclosed subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the presently disclosed subject matter may include various combinations or configurations of presently disclosed features, steps, or elements, or their equivalents (including combinations of features, parts, or steps or configurations thereof not expressly shown in the figures or stated in the detailed description of such figures). Additional embodiments of the presently disclosed subject matter, not necessarily expressed in the summarized section, may include and incorporate various combinations of aspects of features, components, or steps referenced in any summarized objects herewith, and/or other features, components, or steps as otherwise discussed in this application. Those of ordinary skill in the art will better appreciate the features and aspects of such embodiments, and others, upon review of the remainder of the specification, and will appreciate that the presently disclosed subject matter applies equally to corresponding methodologies as associated with practice of any of the present exemplary devices, and vice versa.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIGS. 1(A) through I(F) illustrate left ventricle geometry, with a reference configuration used to calculate myocardial strain, in accordance with presently disclosed exemplary subject matter, and in which FIG. 1(A) illustrates an image during end-systolic imaging, augmented by regional indications, associated with exemplary regional measures, and FIG. 1(D) illustrates an image during end-diastolic imaging, augmented by regional indications, associated with exemplary regional measures, while FIGS. 1(B) and 1(C) graphically illustrate exemplary regional measures of end-systolic radius (FIG. 1(B)) and end-diastolic radius (FIG. 1(C)), respectively, and while FIGS. 1(E) and 1(F) graphically illustrate exemplary regional measures of end-systolic wall thickness (FIG. 1(E)) and wall thickness radius (FIG. 1(F)), respectively, all of the foregoing in accordance with presently disclosed exemplary subject matter;

FIGS. 2(A) through 2(D) graphically illustrate calculated myocardial strain and stress associated with left ventricle geometry, in accordance with presently disclosed exemplary subject matter, and in particular graphically illustrating Radial Strain (FIG. 2(A)), Circumferential Strain (FIG. 2(B)), Radial Stress (FIG. 2(C)), and Circumferential Stress (FIG. 2(D));

FIGS. 3(A) and 3(B) graphically illustrate calculated myocardial radial and circumferential wall stiffness, respectively, associated with left ventricle geometry, in accordance with presently disclosed exemplary subject matter;

FIG. 3(C) graphically illustrates compressive stress data versus compressive strain data while FIG. 3(D) graphically illustrates radial stiffness determined via ex-vivo testing versus radial stiffness determined via speckle tracking echocardiography (STE), in accordance with presently disclosed subject matter;

FIGS. 4(A) through 4(D) relate to clinical translation of LV myocardial stiffness estimation, in accordance with presently disclosed subject matter, with FIG. 4(A) illustrating a representative image of a short-axis echocardiographic view obtained from routine clinical assessment, with the posterior LV myocardium identified as the region of interest (ROI) for strain measure, with FIG. 4(B) graphically illustrating STE-based radial (left) and circumferential (right) LV myocardial strains, as automatically defined by STE software (i.e. end-diastolic to end-systolic strain), and with FIGS. 4(C) and 4(D) graphically representing STE-based posterior region LV myocardial stiffness in the (C) radial and (D) circumferential directions, respectively, computed for each of a non-HFpEF and HFpEF patient;

FIG. 5 illustrates six images of Segment Divided echocardiograph imagery in respective end systolic and end diastolic configurations;

FIGS. 6(A) through 6(F) graphically illustrate strain over a single heartbeat in the radial and circumferential directions, for the respective six images of FIG. 5;

FIGS. 7(A) through 7(F) illustrate images of Short Axis Echocardiograph Imagery of the Left Ventricle at the Level of the Papillary Muscles of respective targets, particularly showing LV images at end diastole for a Recellularization (RC), left ventricular pressure overload (LVPO), and exercised pigs in FIGS. 7(A), 7(B), and 7(C), respectively, and showing LV images of end systole for a Recellularization (RC), left ventricular pressure overload (LVPO), and exercised pigs in FIGS. 7(D), 7(E), and 7(F), respectively; and

FIG. 8 illustrating a representative image of an annotated Echocardiograph Image from end diastole of the left ventricular pressure overload (LVPO) pig.

Repeat use of reference characters in the present specification and drawings is intended to represent the same or analogous features, elements, or steps of the presently disclosed subject matter.

### DETAILED DESCRIPTION OF PRESENTLY DISCLOSED SUBJECT MATTER

Reference will now be made in detail to various embodiments of the disclosed subject matter, one or more examples of which are set forth herein. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

### 2. Methods

### 2.1 Study Groups

We demonstrate the presently disclosed protocol using representative echocardiographic (STE) data obtained from previous preclinical (porcine) studies of LV (left ventricle) remodeling; specifically, we consider an animal exhibiting maladaptive LV remodeling due to progressive left ventricular pressure overload (LVPO) (Torres et al., 2020), an animal exhibiting adaptive LV remodeling upon completion of an exercise protocol (Samani et al., 2024), and an age-matched referent control animal (Torres et al., 2020). The presently disclosed focus on pressure-mediated maladaptive LV remodeling is clinically motivated by its central role in the development of HFpEF (heart failure (HF) with preserved ejection fraction), wherein a primary output of the remodeling process is an increase in LV myocardial stiffness (Grossman et al., 1974; Jalil et al., 1989; Torres et al., 2020; Yarbrough et al., 2012). Moreover, because LVPO induces concentric and hypertrophic remodeling (McMullen and Jennings, 2007), we expect a reasonable correspondence between actual LV geometry and a simple geometrical model that facilitates estimation of LV myocardial stress (as detailed below). We include an exercised animal in this protocol demonstration for both comparative purposes and the potential role of exercise in cardiac therapies (Edelmann et al., 2011; Lear et al, 2017). Indeed, LV remodeling in response to exercise is also concentric and hypertrophic (McMullen and Jennings, 2007), but this process is adaptive due to a concurrent reduction in LV myocardial stiffness and preservation/reduction of LV chamber stiffness (Samani et al., 2024).

In addition to geometric considerations (instances of concentric and hypertrophic remodeling), the presently disclosed protocol is further specified for assessment of LV myocardial stiffness as manifest in the diastolic phase of the cardiac cycle. Therefore, obtained stiffness measures are most relevant to the passive LV myocardial behavior (and thus reflect the effects of cardiac fibrosis prototypical of HFpEF) as opposed to LV myocardial contractility (which is more relevant to systolic HF modalities).

Next, to support the translational feasibility of LV myocardial stiffness estimation, we demonstrate the presently disclosed protocol on routine clinical images obtained from both a non-HFpEF and a HFpEF patient. Both patients have diabetes managed with oral agents and had been completely revascularized for multivessel coronary heart disease. The patient with HFpEF underwent surgical replacement of his aortic valve 2 years prior to the echocardiogram and had persistent Class 2 HF symptoms managed with 1 mg bumetanide daily. Measured LV end-diastolic pressure was 18 mmHg, H2FPEF score was 4 (BMI-34.2, Hypertension, E/e′>9), and brain natriuretic peptide (BNP) was 82 pg/mL. The patient without HFpEF was asymptomatic, LV end-diastolic pressure was 6 mmHg, and H2FPEF score was 0.

### 2.2 Mechanical Model: Applicability, Assumptions, and Governing Equations

The presently disclosed protocol is specified for assessment of LV myocardial stiffness as manifest in the diastolic phase of the cardiac cycle. Therefore, obtained stiffness measures are most relevant to the passive LV myocardial behavior (and thus reflect the effects of cardiac fibrosis prototypical of HFpEF) as opposed to LV myocardial contractility (which is more relevant to systolic HF modalities).

Guided by these protocol objectives and specifications, we focus on the LV short-axis view at the level of the papillary muscle and consider the mid-wall LV myocardial deformation from end-systole (ES, reference configuration) to the end-diastole (ED, deformed configuration). Regional myocardial strains (ε) are thus defined via the general relation:

$$ε = \frac{{l}_{ED} - {l}_{ES}}{{l}_{ES}} , \qquad ( 1 )$$

where l_{ED} and l_{ES} are the lengths of a regionally-contained segment at ED and ES, respectively, with segment and associated strain orientations in either the radial (εᵣ) or circumferential (ε_{θ}) direction. Note that although typical STE analytical software considers ED as the reference configuration and computes a segmental strain at ES, a simple transformation of obtained measures can be applied to yield the strain as defined in Eq. (1).

For example, given a typical STE strain output (ε^{*}) defined as

$${ε}^{*} = \frac{{l}_{E S} - {l}_{E D}}{{l}_{E D}} , then ε = \frac{- {ε}^{*}}{( 1 + {ε}^{*} )} .$$

In the LV short-axis view, the kinematics and geometry of diastolic LV myocardial deformation in the considered cases (referent control or after concentric and hypertrophic LV remodeling) can be reasonably modeled as a thick-walled cylinder that is inflated by an internal pressure. In the reference configuration, we assume the LV chamber pressure is zero; in the deformed configuration, we assume the LV chamber pressure is equal to the pulmonary capillary wedge pressure (PCWP). We ignore the presence of residual strains in the LV myocardium, and thus assume the traction-free (zero pressure) reference configuration is also a zero-stress/zero-strain configuration.

Out-of-plane myocardial deformation (i.e. twisting) and associated shear strains are not accounted for in the presently disclosed protocol, which is an inherent limitation of the obtained values for LV myocardial stiffness.

To estimate mean regional wall stress (σ) in the deformed configuration, we apply the universal solutions for a uniformly inflated thick-walled circular cylinder at mechanical equilibrium. Within a specified LV myocardial region (for which STE-based strains have been measured), we compute the associated mean LV myocardial stresses in the radial (σᵣ) and circumferential (σ_{θ}) directions as

$${σ}_{r} = - \frac{P}{2} and {σ}_{θ} = \frac{{Pr}_{i}}{t} , \qquad ( 2 )$$

where P is the LV chamber pressure; rᵢ is the deformed inner radius of the LV; t is the deformed LV myocardial wall thickness. Note that these expressions for mean stresses are derived for a circular cylindrical cross-section with a traction-free outer surface in a state of axisymmetric deformation, and their use despite potential regional variations in geometry and outer surface boundary conditions (i.e. free wall vs. septal wall) yields only approximate values. While σᵣ only depends on the mechanical load, regional variations in LV myocardial geometry are explicitly accounted for in computed values for σ_{θ}.

Finally, we assume that the slope of the linearized regional stress/strain relation when going from the reference to the deformed configuration developed in either direction can be considered as an estimate of regional diastolic LV myocardial stiffness (K_{M}). Thus, the regional LV myocardial stiffness in the radial (K_{M,r}) and circumferential (K_{M,θ}) directions are computed via ED stress/strain ratios, namely

$${K}_{M , r} = \frac{{σ}_{r}}{{ε}_{r}} and {K}_{M , θ} = \frac{{σ}_{θ}}{{ε}_{θ}} . \qquad ( 3 )$$

Given the kinematics of diastolic deformation, K_{M,r} and K_{M,θ} provide stiffness estimates for the LV myocardium under compression and tension, respectively.

### 3. Results and Discussion

### 3.1 Preclinical Examples

All animals were treated and cared for in accordance with the National Institutes of Health Guide for the Care and Use of Laboratory Animals (Eighth Edition. Washington, DC: 2011), and all protocols were approved by the University of South Carolina School of Medicine and WJB Dorn VA Institutional Animal Care and Use Committee. To minimize pain and distress during non-invasive echocardiographic studies, animals were administered oral diazepam (200 mg) one hour prior to the imaging procedure and supplemental midazolam (0.5-0.6 mg/kg) intramuscularly at the time of the echocardiogram.

We illustrate the presently disclosed protocol for estimation of LV myocardial stiffness with short-axis LV echocardiograms (GE Vivid E9 with XDclear Ultrasound System: M5S [1.5 to 4.6 Hz] transducer probe; GE, Boston, MA) on three representative, age-matched animals extracted from previous studies on LV remodeling (referent control, LVPO, and exercise, as described above). First, we used a commercial echocardiographic analysis software (EchoPAC™) and established methods to measure regional LV wall geometry (wall thickness and inner radius) at ES and ED, as well as conventional Doppler methods to measure PCWP (Nagueh et al., 1997). In all cases, regional wall geometry in both the reference (ES) and deformed (ED) configurations is relatively uniform (as indicated by the standard deviation of the regional average of each measure), supporting the employed geometric/kinematic model of diastolic LV deformation (the uniform inflation of a thick-walled cylindrical tube).

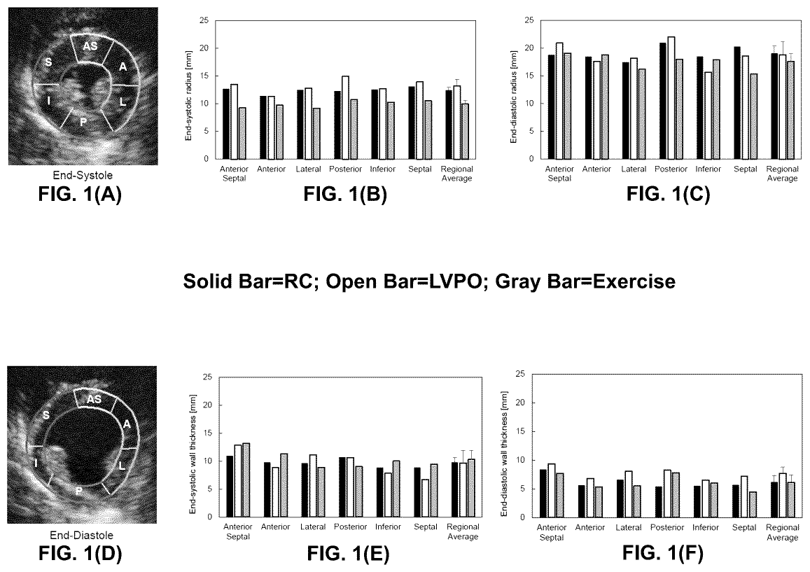

In particular, FIGS. 1(A) through I(F) illustrate left ventricle (LV) geometry, with a reference configuration used to calculate myocardial strain, in accordance with presently disclosed exemplary subject matter. More particularly, FIG. 1(A) illustrates an image during end-systolic imaging, augmented by regional indications, associated with exemplary regional measures. Similarly, FIG. 1(D) illustrates an image but during end-diastolic imaging, augmented by regional indications, associated with exemplary regional measures. FIGS. 1(B) and 1(C) graphically illustrate (bar graphs) exemplary regional measures of end-systolic radius (FIG. 1(B)) and end-diastolic radius (FIG. 1(C)), respectively. FIGS. 1(E) and 1(F) graphically illustrate (bar graphs) exemplary regional measures of end-systolic wall thickness (FIG. 1(E)) and wall thickness radius (FIG. 1(F)), respectively, all of the foregoing in accordance with presently disclosed exemplary subject matter.

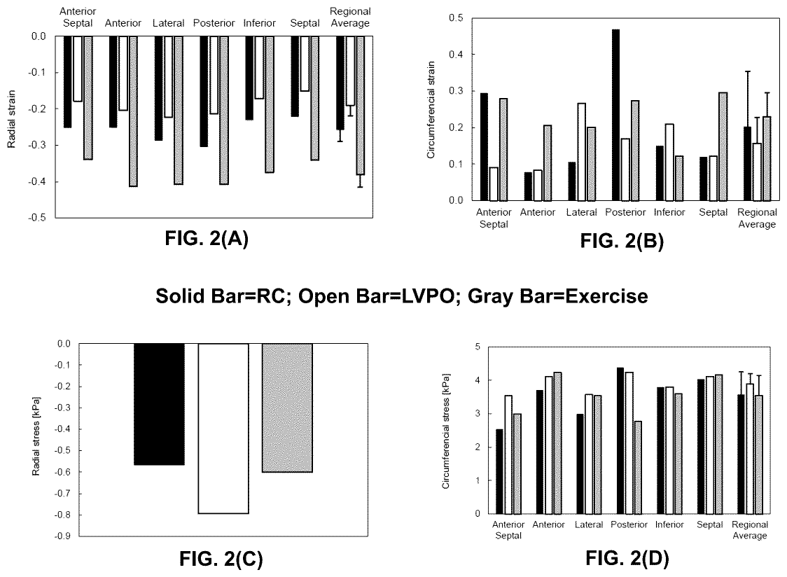

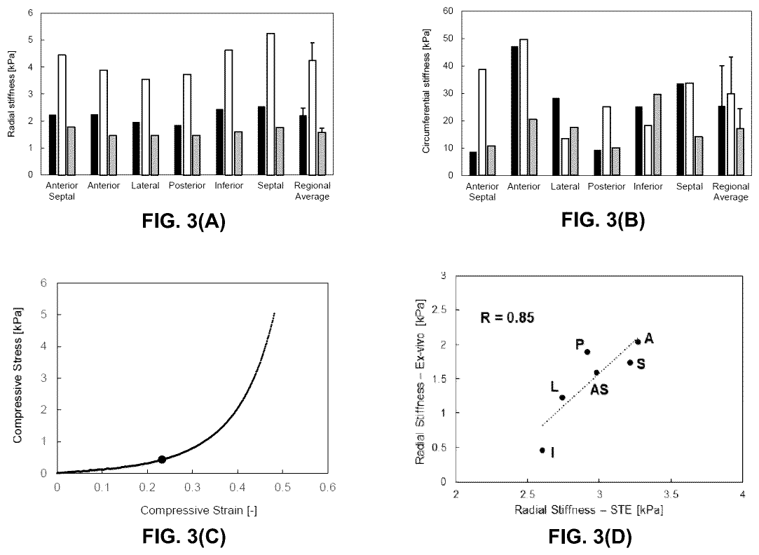

Thus, FIGS. 1(A) and 1(D) relate to left ventricle (LV) geometry, including reference and deformed configurations, respectively. Specification and quantification of the reference and deformed LV configurations are used for estimation of LV myocardial stiffness. The reference configuration used to calculate myocardial strain is characterized by regional measures of end-systolic radius (FIG. 1(B)) and wall thickness (FIG. 1(E)), while the deformed configuration is characterized by regional measures of end-diastolic radius (FIG. 1(C)) and wall thickness (FIG. 1(F)). STE-based definition of LV myocardial regions in the short-axis echocardiographic view include referent control animal; AS—anterior septal; A—anterior; L—lateral; P—posterior; I—inferior; S—septal; referent control; and corresponding measures of LV chamber radius and myocardial wall thickness for the reference (FIGS. 1(A)-1(C)) and deformed (FIGS. 1(D)-1(F)) LV configurations. In the bar graphs of FIGS. 1(B), 1(C), 1(E), and 1(F), the solid bar columns refer to referent control animal; the white (or open) columns refer to LVPO animal, and the gray columns refer to exercised subjects. The same designations of data listings are used in the bar graphs of FIGS. 2(A)-2(D) and FIGS. 3(A) and 3(B). In line with the presently disclosed protocol, regional radial and

circumferential LV myocardial strains (Eq. 1), mean LV myocardial stress (Eq. 2), and corresponding estimates of LV myocardial stiffness (Eq. 3) were computed for a referent control and LVPO animal. In particular, FIGS. 2(A) through 2(D) graphically illustrate (bar graphs) calculated myocardial strain and stress associated with left ventricle geometry, in accordance with presently disclosed exemplary subject matter, and in particular graphically illustrating Radial Strain (FIG. 2(A)), Circumferential Strain (FIG. 2(B)), Radial Stress (FIG. 2(C)), and Circumferential Stress (FIG. 2(D)). FIGS. 3(A) and 3(B) graphically illustrate (bar graphs) calculated myocardial radial and circumferential wall stiffness, respectively, associated with left ventricle geometry, in accordance with presently disclosed exemplary subject matter.

Comparison between obtained values of K_{M,r} (FIG. 3A) and K_{M,θ} (FIG. 3B) in these representative animals suggests the presently disclosed protocol can detect the regional and directional variance in LV myocardial stiffness, as well as the expected increase in LV myocardial stiffness with LVPO. Moreover, the generally order-of-magnitude difference between K_{M,r} and K_{M,θ} reflects the expected anisotropic behavior of the LV myocardium, with the comparatively high values of K_{M,θ} due to the characteristic behavior of the primary load-bearing wall constituent (collagen) under tension.

To validate STE-based estimation of LV myocardial stiffness, we compared computed K_{M,r} values to analogous values derived from regionally- and directionally-matched ex-vivo mechanical testing of LV myocardium. Following established mechanical testing protocols (Shazly et al., 2008), LV myocardial samples were obtained the day following echocardiographic studies on a referent control animal, prepared as cylindrical test elements (˜5 mm diameter; 3 mm length), and subjected to an unconfined radial compression test (ramped uniaxial displacement; displacement rate of 0.05 mm/sec; total displacement of ˜1.5 mm) using a mechanical testing apparatus appropriated for soft tissue analyses (Bose® Biodynamic Test Instrument, Minnetonka, MN). Resultant force and displacement data were continuously recorded (data scan rate of 200/sec) by the system load cell and software package (WinTest® Software, Minnetonka, MN) and transformed to yield a compressive stress-strain relation (FIG. 3(C)). The stress-strain coordinate corresponding to the STE-derived εᵣ was identified for each LV myocardial region (solid data point in FIG. 3(D)) and subsequently used to calculate radial LV myocardial stiffness per Eq. 3. In particular, FIG. 3(C) graphically illustrates compressive stress data versus compressive strain data while FIG. 3(D) graphically illustrates radial stiffness determined via ex-vivo testing versus radial stiffness determined via speckle tracking echocardiography (STE), in accordance with presently disclosed subject matter. In other words, regional averages and standard deviations are included for all measures with regional variation regarding FIGS. 3(A) and 3(B). FIG. 3(C) provides data on representative compressive stress-strain response from ex-vivo uniaxial testing of referent control LV myocardium, where the solid circle data point represents relevant data for isometric comparison with STE-based LV myocardial stiffness estimates. FIG. 3(D) provides data for correlation between regional LV myocardial stiffness estimates derived via ex-vivo uniaxial mechanical testing and STE-based analysis (referent control animal; AS—anterior septal; A—anterior; L—lateral; P—posterior; I—inferior; S—septal). Isometric comparison of K_{M,r} resulting from these techniques suggests a systematic overestimation of LV myocardial stiffness with STE when compared to ex-vivo testing (regional mean K_{M,r} of 2.9 kPa vs 1.5 kPa, respectively), but excellent correlation (R=0.85) among regional values supports the diagnostic potential of the presently disclosed STE-based protocol (FIG. 2H).

### 3.1 Clinical Examples

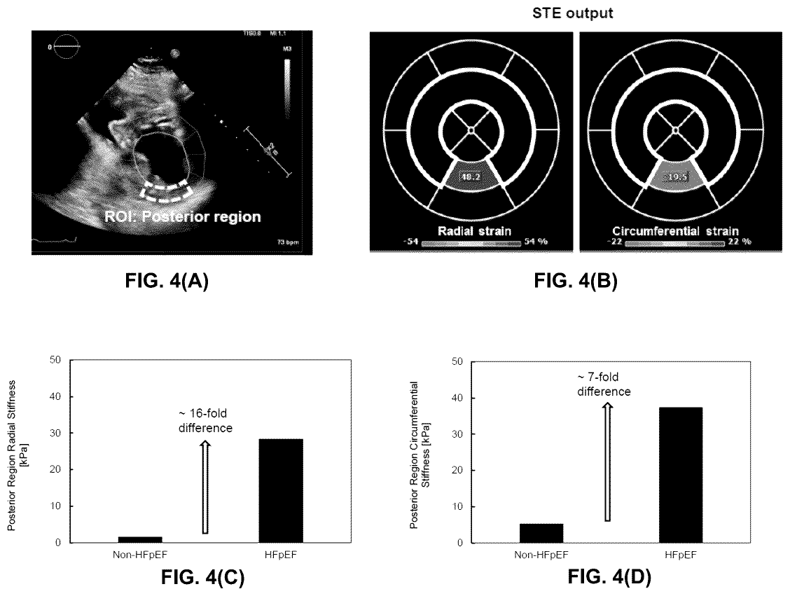

Routine short-axis echocardiographic imaging (FIG. 4A) and STE-based measures of LV myocardial strains (FIG. 4B) were obtained for a non-HFpEF and HFpEF patient (as described above). Based on available image-quality and resultant speckle tracking, mechanical/geometrical measurements were restricted to the posterior LV myocardial region for the purpose of demonstrating the translational feasibility of the presently disclosed protocol. STE-based estimation of LV myocardial stiffness shows notable differences between non-HFpEF and HFpEF patients, with approximate 16- and 7-fold differences observed in the radial (FIG. 4(C)) and circumferential (FIG. 4(D)) directions, respectively.

In particular, FIGS. 4(A) through 4(D) relate to clinical translation of LV myocardial stiffness estimation, in accordance with presently disclosed subject matter. Further, FIG. 4(A) illustrates a representative image of a short-axis echocardiographic view obtained from routine clinical assessment, with the posterior LV myocardium identified as the region of interest (ROI) for strain measure. FIG. 4(B) graphically illustrates STE-based radial (left) and circumferential (right) LV myocardial strains, as automatically defined by STE software (i.e. end-diastolic to end-systolic strain). FIGS. 4(C) and 4(D) graphically representing STE-based posterior region LV myocardial stiffness in the (C) radial and (D) circumferential directions, respectively, computed for each of a non-HFpEF and HFpEF patient.

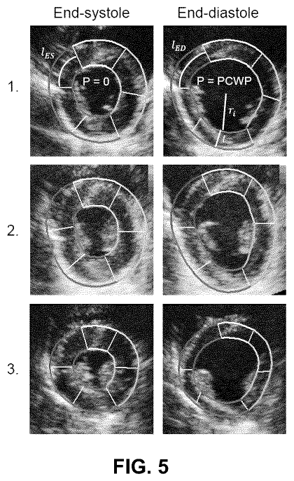

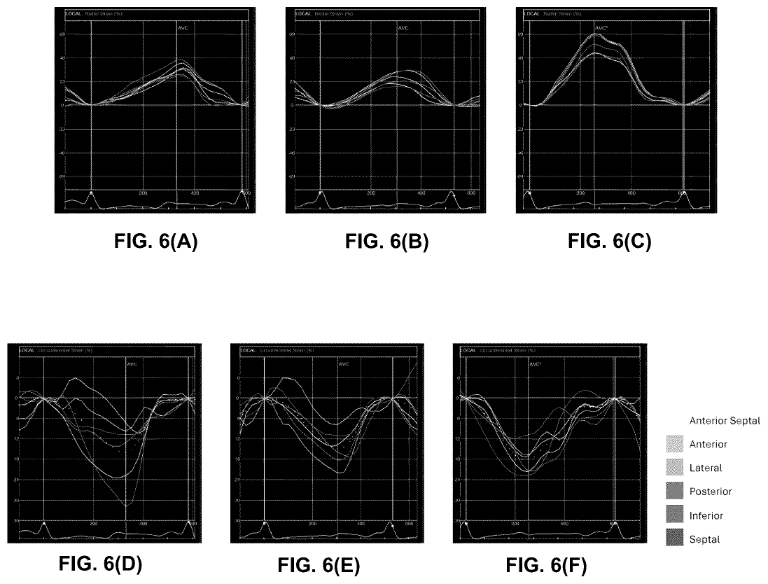

FIG. 5 illustrates six images of Segment Divided echocardiograph imagery in respective end systolic and end diastolic configurations, in the left and right columns, respectively. Images associated with RC, LVPO, and Exercised Pigs are represented by rows 1, 2, and 3 respectively in both left and right side columns. FIGS. 6(A) through 6(F) graphically illustrate strain over a single heartbeat in the radial and circumferential directions, for the respective six images of FIG. 5. In particular, in images of row 1 of the left hand and right hand columns of FIG. 5, echocardiograph images are annotated to display measurements used in calculations, in accordance with presently disclosed subject matter.

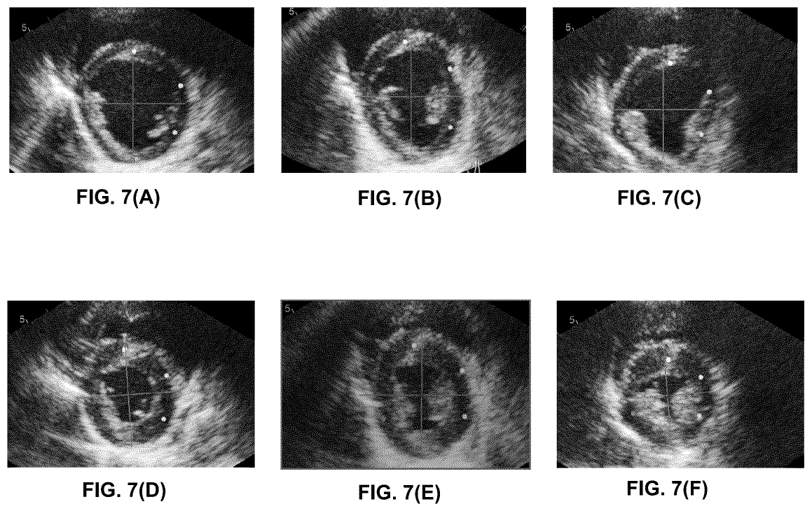

FIGS. 7(A) through 7(F) illustrate images of Short Axis Echocardiograph Imagery of the Left Ventricle at the Level of the Papillary Muscles of respective targets, particularly showing LV images at end diastole for a Recellularization (RC), left ventricular pressure overload (LVPO), and exercised pigs in FIGS. 7(A), 7(B), and 7(C), respectively, and showing LV images of end systole for a Recellularization (RC), left ventricular pressure overload (LVPO), and exercised pigs in FIGS. 7(D), 7(E), and 7(F), respectively. Stated another way, such FIGS. 7(A) through 7(F) illustrate myocardial wall strain over one heartbeat, with raw strain output before mathematical transformation, and with end diastole as zero reference point. Strain is shown in the radial direction in connection with FIGS. 7(A)-7(C), while strain is shown in the circumferential direction in connection with FIGS. 7(D)-7(F).

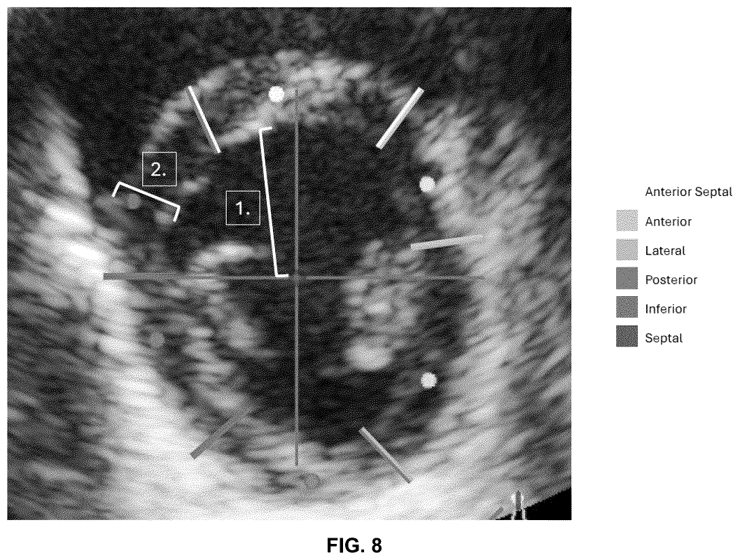

FIG. 8 illustrates a representative image of an annotated Echocardiograph Image from end diastole of the left ventricular pressure overload (LVPO) pig specimen. Per the annotations, boarders of the regions of myocardium are shown by gray-scale bars, while indicated Distance 1 shows the radius of the anterior septal region and indicated Distance 2 shows the wall thickness of the septal region.

### 4. Conclusion

We present a protocol for processing of STE-derived LV myocardial strain data to generate estimates of LV myocardial stiffness. Maladaptive LV remodeling is a canonical process in HFpEF, wherein increased LV myocardial stiffness contributes to disease progression and insomuch represents a potentially valuable response variable to improve clinical decision making. This protocol demonstration details a novel methodology for estimating LV myocardial stiffness in both a preclinical and clinical setting; subsequent studies will be performed to (i) further validate obtained LV myocardial stiffness estimates (via comparison with ex-vivo measures of LV myocardial stiffness in a preclinical model) and (ii) further establish the clinical utility of estimating LV myocardial stiffness (via correlation between LV myocardial stiffness, LV chamber stiffness, and HFpEF development/progression in patients).

While certain embodiments of the disclosed subject matter have been described using specific terms, such description is for illustrative purposes only, and it is to be understood that changes and variations may be made without departing from the spirit or scope of the subject matter.

### REFERENCES

Bansal, M. Kasliwal, R. R., 2013. How do I do it? Speckle-tracking echocardiography. Indian Heart Journal 1, 117-123.
Behnoush, A. H. Khalaji, A. Naderi, N. Ashraf, H. von Haehling, S., 2023 ACC/AHA/HFSA 2022 and ESC 2021 guidelines on heart failure comparison. ESC Heart Failure 10, 1531-1544.
Chan, J. Hanekom, L. Wong, C. Leano, R. Cho, G. Y. Marwick, T. H., 2006. Differentiation of subendocardial and transmural infarction using two-dimensional strain rate imaging to assess short-axis and long-axis myocardial function. Journal of the American College of Cardiology 48, 2026-2033.
Collier, P. Phelan, D. Klein, A., 2017. A Test in Context: Myocardial Strain Measured by Speckle-Tracking Echocardiography. Journal of the Americal College of Cardiology 69, 1043-1056.
Edelmann, F. Gelbrich, G. Düngen, H. D. Fröhling, S. Wachter, R. Stahrenberg, R. Binder, L. Topper, A. Lashki, D. J. Schwarz, S. Herrmann-Lingen, C. Löffler, M. Hasenfuss, G. Halle, M. Pieske, B., 2011. Exercise training improves exercise capacity and diastolic function in patients with heart failure with preserved ejection fraction: results of the Ex-DHF (Exercise training in Diastolic Heart Failure) pilot study. Journal of the American College of Cardiology 58, 1780-1791.
Gjesdal, O. Helle-Valle, T. Hopp, E. Lunde, K. Vartdal, T. Aakhus, S. Smith, H. J. Ihlen, H. Edvardsen, T., 2008. Noninvasive separation of large, medium, and small myocardial infarcts in survivors of reperfused ST-elevation myocardial infarction: a comprehensive tissue Doppler and speckle-tracking echocardiography study. Circulation: Cardiovascular Imaging 1, 189-196.
Grossman, W. McLaurin, L. P. Stefadouros, M. A., 1974. Left ventricular stiffness associated with chronic pressure and volume overloads in man. Circulation Research 35, 793-800
Haugaa, K. H. Grenne, B. L. Eek, C. H. Ersbøll, M. Valeur, N. Svendsen, J. H. Florian, A. Sjøli, B. Brunvand, H. Køber, L. Voigt, J. U. Desmet, W. Smiseth, O. A. Edvardsen, T., 2013. Strain echocardiography improves risk prediction of ventricular arrhythmias after myocardial infarction. Journal of the Americal College of Cardiology: Cardiovascular Imaging 6, 841-850.
Heidenreich, P. A. Bozkurt, B. Aguilar, D. Allen, L. A. Byun, J. J. Colvin, M. M. Deswal, A. Drazner, M. H. Dunlay, S. M. Evers, L. R. Fang, J. C. Fedson, S. E. Fonarow, G. C. Hayek, S. S. Hernandez, A. F. Khazanie, P. Kittleson, M. M. Lee, C. S. Link, M. S. Milano, C. A. Nnacheta, L. C. Sandhu, A. T. Stevenson, L. W. Vardeny, O. Vest, A. R. Yancy, C. W., 2022. 2022 AHA/ACC/HFSA Guideline for the Management of Heart Failure: A Report of the American College of Cardiology/American Heart Association Joint Committee on Clinical Practice Guidelines. Circulation 145, 895-1032.
Jalil, J. E. Doering, C. W. Janicki, J. S. Pick, R. Shroff, S. G. Weber, K. T., 1989. Fibrillar collagen and myocardial stiffness in the intact hypertrophied rat left ventricle. Circulation: Research 64, 1041-1050.
Jin, X. Nauta, J. F. Hung, C. L. Ouwerkerk, W. Teng, T. K. Voors, A. A. Lam, C. S. van Melle, J. P., 2022. Left atrial structure and function in heart failure with reduced (HFrEF) versus preserved ejection fraction (HFpEF): systematic review and meta-analysis. Heart Failure Reviews 27. 1933-1955.
Lear, S. A. Hu, W. Rangarajan, S. Gasevic, D. Leong, D. Iqbal, R. Casanova, A. Swaminathan, S. Anjana, R. M. Kumar, R. Rosengren, A. Wei, L. Yang, W. Chuangshi, W. Huaxing, L. Nair, S. Diaz, R. Swidon, H. Gupta, R. Mohammadifard, N. Lopez-Jaramillo, P. Oguz, A. Zatonska, K. Seron, P. Avezum, A. Poirier, P. Teo, K. Yusuf, S., 2017. The effect of physical activity on mortality and cardiovascular disease in 130 000 people from 17 high-income, middle-income, and low-income countries: the PURE study. Lancet 390. 2643-2654.
Li, S. X. Wang, Y. Lama, S. D. Schwartz, J. Herrin, J. Mei, H. Lin, Z. Bernheim, S. M. Spivack, S. Krumholz, H. M. Suter, L. G., 2020. Timely estimation of National Admission, readmission, and observation-stay rates in medicare patients with acute myocardial infarction, heart failure, or pneumonia using near real-time claims data. BioMed Central: Health Services Research 20. 733.
Liu, W. Wang, Z., 2019. Current Understanding of the Biomechanics of Ventricular Tissues in Heart Failure. Bioengineering 7. 2.
McMullen, J. R. Jennings, G. L., 2007. Differences between pathological and physiological cardiac hypertrophy: novel therapeutic strategies to treat heart failure. Clinical and Experimental Pharmacology and Physiology 34. 255-262.
Mehrotra, A. Kacker, S. Shadab, M. Chandra, N. Singh, A. K., 2022. 4 Dimensional XStrain speckle tracking echocardiography: comprehensive evaluation of left ventricular strain and twist parameters in healthy Indian adults during COVID-19 pandemic. American Journal of Cardiovascular Disease 12. 192-204.
Mohebi, R. Chen, C Ibrahim, N. E. McCarthy, C. P. Gaggin, H. K. Singer, D. E. Hyle, E. P. Wasfy, J. H. Januzzi, J. L., 2022. Cardiovascular Disease Projections in the United States Based on the 2020 Census Estimates. Journal of the American College of Cardiology 80. 565-578.
Nagueh, S. F. Middleton, K. J. Kopelen, H. A. Zoghbi, W. A. Quiñones, M. A., 1997. Doppler tissue imaging: a noninvasive technique for evaluation of left ventricular relaxation and estimation of filling pressures. Journal of the American College of Cardiology 30. 1527-1533.
Obokata, M. Reddy, Y. N. V. Borlaug, B. A., 2020. Diastolic Dysfunction and Heart Failure With Preserved Ejection Fraction: Understanding Mechanisms by Using Noninvasive Methods. Journal of the American College of Cardiology: Cardiovascular Imaging 13, 245-257.
Omens, J. H. Fung, Y. C., 1990. Residual strain in rat left ventricle. Circulation Research 66. 37-45
Omote, K. Verbrugge, F. H. Borlaug, B. A., 2022. Heart Failure with Preserved Ejection Fraction: Mechanisms and Treatment Strategies. Annual Review of Medicine 73. 321-337.
Reant, P. Labrousse, L. Lafitte, S. Bordachar, P. Pillois, X. Tariosse, L. Bonoron-Adele, S. Padois, P. Deville, C. Roudaut, R. Dos Santos, P., 2008. Experimental validation of circumferential, longitudinal, and radial 2-dimensional strain during dobutamine stress echocardiography in ischemic conditions. Journal of the American College of Cardiology 51. 149-157.
Redfield, M. M. Borlaug, B. A., 2023. Heart Failure With Preserved Ejection Fraction: A Review. Journal of the American Medical Association 329. 827-838.
Sakata, Y. Ohtani, T. Takeda, Y. Yamamoto, K. Mano, T., 2013. Left ventricular stiffening as therapeutic target for heart failure with preserved ejection fraction. Circulation Journal 77. 886-892.
Samani, S. L. Barlow, S. C. Freeburg, L. A. Jones, T. L. Poole, M. Sarzynski, M. A. Zile, M. R. Shazly, T. Spinale, F. G., Left ventricle function and post-transcriptional events with exercise training in pigs. PLOS ONE 19. e0292243.
Stanton, T. Leano, R. Marwick, T. H., 2009. Prediction of all-cause mortality from global longitudinal speckle strain: comparison with ejection fraction and wall motion scoring. Circulation Cardiovascular Imaging 2. 356-354.
Sun, J. P. Niu, J. Chou, D. Chuang, H. H. Wang, K. Drinko, J. Borowski, A. Stewart, W. J. Thomas, J. D., 2007. Alterations of regional myocardial function in a swine model of myocardial infarction assessed by echocardiographic 2-dimensional strain imaging. Journal of the American Society of Echocardiography 20. 498-504.
Torres, W. M. Barlow, S. C. Moore, A. Freeburg, L. A. Hoenes, A. Doviak, H. Zile, M. R. Shazly, T. Spinale, F. G., 2020. Changes in Myocardial Microstructure and Mechanics With Progressive Left Ventricular Pressure Overload. Journal of the America College of Cardiology: Basic to Translational Science 5. 463-480.
Torres, W. M. Jacobs, J. Doviak, H. Barlow, S. C. Zile, M. R. Shazly, T. Spinale, F. G., 2018. Regional and temporal changes in left ventricular strain and stiffness in a porcine model of myocardial infarction. American Journal of Physiology-Heart and Circulatory Physiology 4. H958-H967.
Yarbrough, W. M. Mukherjee, R. Stroud, R. E. Rivers, W. T. Oelsen, J. M. Dixon, J. A. Eckhouse, S. R. Ikonomidis, J. S. Zile, M. R. Spinale, F. G., 2012. Progressive induction of left ventricular pressure overload in a large animal model elicits myocardial remodeling and a unique matrix signature. Journal of Thoracic and Cardiovascular Surgery 143. 215-223.

What is claimed is:

1. Methodology for using echocardiography imaging to evaluate patient heart failure risk for guiding treatment protocols, comprising:
acquiring and processing echocardiographic imaging data for the left ventricle (LV) of a patient's heart;
using the imaging data to calculate regional LV myocardial strains;
estimating mean regional LV wall stress by calculating the mean LV myocardial stresses associated with the calculated regional LV myocardial strains;
using the calculated regional LV myocardial strains and associated LV myocardial stresses to estimate regional LV myocardial stiffness; and
using the estimated regional LV myocardial stiffness to gauge development and progression of patient heart failure.

2. Methodology according to claim 1, wherein the echocardiography imaging comprises two-dimensional speckle tracking echocardiography (STE).

3. Methodology according to claim 1, wherein:
the echocardiography imaging comprises short axis echocardiograph imagery of the LV at the level of the papillary muscles of respective targets;
calculating regional LV myocardial strains (ε) includes imaging mid-wall LV myocardial deformation from end-systole (ES) to the end-diastole (ED).

4. Methodology according to claim 3, wherein the regional myocardial strains (ε) are calculated per the equation:
$$ε = \frac{{l}_{ED} - {l}_{ES}}{{l}_{ES}} ,$$
where l_{ED} and l_{ES} are the lengths of a regionally-contained segment at ED and ES, respectively, with segment and associated strain orientations in either the radial (εᵣ) or circumferential (ε_{θ}) direction.

5. Methodology according to claim 4, further comprising transforming calculated regional myocardial strains (ε) to a strain output (ε^{*}) defined as
$${ε}^{*} = \frac{{l}_{E S} - {l}_{E D}}{{l}_{E D}} , with ε = \frac{- {ε}^{*}}{( 1 + {ε}^{*} )} .$$

6. Methodology according to claim 3, wherein estimating mean regional LV wall stress (σ) comprises computing the associated mean LV myocardial stresses in the radial (σᵣ) and circumferential (σ_{θ}) directions as
$${σ}_{r} = - \frac{P}{2} and {σ}_{θ} = \frac{{Pr}_{i}}{t} ,$$
respectively,
where P is the LV chamber pressure; rᵢ is the deformed inner radius of the LV; and t is the deformed LV myocardial wall thickness.

7. Methodology according to claim 6, wherein estimating regional LV myocardial stiffness (K_{M}) includes using the slope of the linearized regional stress/strain relation.

8. Methodology according to claim 7, wherein the regional LV myocardial stiffness in the radial (K_{M,r}) and circumferential (K_{M,θ}) directions are computed via ED stress/strain ratios, respectively,
$${K}_{M , r} = \frac{{σ}_{r}}{{ε}_{r}} and {K}_{M , θ} = \frac{{σ}_{θ}}{{ε}_{θ}} .$$

9. Methodology according to claim 7, wherein the regional LV myocardial stiffness in the radial (K_{M,r}) and circumferential (K_{M,θ}) directions are computed via ED stress/strain ratios, respectively, to provide stiffness estimates for the LV myocardium under compression and tension, respectively.

10. Methodology according to claim 1, wherein:
the echocardiography imaging comprises echocardiograph imagery of the LV at the level of the papillary muscles of respective targets;
calculating regional LV myocardial strains (ε) includes imaging mid-wall LV myocardial deformation from end-systole (ES) to the end-diastole (ED);
the regional myocardial strains (ε) are calculated per the equation:
$$ε = \frac{{l}_{ED} - {l}_{ES}}{{l}_{ES}} ,$$
where l_{ED} and l_{ES} are the lengths of a regionally-contained segment at ED and ES, respectively, with segment and associated strain orientations in either the radial (εᵣ) or circumferential (ε_{θ}) direction;
estimating mean regional LV wall stress (σ) comprises computing the associated mean LV myocardial stresses in the radial (σᵣ) and circumferential (σ_{θ}) directions as
$${σ}_{r} = - \frac{P}{2} and {σ}_{θ} = \frac{{Pr}_{i}}{t} ,$$
respectively,
where P is the LV chamber pressure; rᵢ is the deformed inner radius of the LV; and t is the deformed LV myocardial wall thickness; and
wherein estimating regional LV myocardial stiffness (K_{M}) includes calculating the radial (K_{M,r}) and circumferential (K_{M,θ}) directions via ED stress/strain ratios, respectively,
$${K}_{M , r} = \frac{{σ}_{r}}{{ε}_{r}} and {K}_{M , θ} = \frac{{σ}_{θ}}{{ε}_{θ}} .$$

11. Methodology for using short-axis left ventricle (LV) echocardiograms for estimating LV myocardial stiffness echocardiography imaging to evaluate patient heart failure risk for guiding treatment protocols, comprising:
acquiring and processing short-axis LV echocardiographic imaging data for the LV at the level of the papillary muscles of respective targets of a patient's heart;
calculating regional LV myocardial strains (ε) includes imaging mid-wall LV myocardial deformation from end-systole (ES) to the end-diastole (ED);
estimating mean regional LV wall stress (σ) by calculating the mean LV myocardial stresses associated with the calculated regional LV myocardial strains;
estimating regional LV myocardial stiffness (K_{M}) by using the slope of the linearized regional stress/strain relation from the calculated regional LV myocardial strains and associated LV myocardial stresses; and
using the estimated regional LV myocardial stiffness to gauge development and progression of patient heart failure.

12. Methodology according to claim 11, wherein:
calculating regional LV myocardial strains (ε) includes imaging mid-wall LV myocardial deformation from end-systole (ES) to the end-diastole (ED);
the regional myocardial strains (ε) are calculated per the equation:
$$ε = \frac{{l}_{ED} - {l}_{ES}}{{l}_{ES}} ,$$
where l_{ED} and l_{ES} are the lengths of a regionally-contained segment at ED and ES, respectively, with segment and associated strain orientations in either the radial (εᵣ) or circumferential (ε_{θ}) direction;
estimating mean regional LV wall stress (σ) comprises computing the associated mean LV myocardial stresses in the radial (σᵣ) and circumferential (σ_{θ}) directions as
$${σ}_{r} = - \frac{P}{2} and {σ}_{θ} = \frac{{Pr}_{i}}{t} ,$$
respectively,
where P is the LV chamber pressure; rᵢ is the deformed inner radius of the LV; and t is the deformed LV myocardial wall thickness; and
wherein estimating regional LV myocardial stiffness (K_{M}) includes calculating the radial (K_{M,r}) and circumferential (K_{M,θ}) directions via ED stress/strain ratios, respectively,
$${K}_{M , r} = \frac{{σ}_{r}}{{ε}_{r}} and {K}_{M , θ} = \frac{{σ}_{θ}}{{ε}_{θ}} .$$

13. Methodology for using imaging to estimate soft tissue conditions within the body for evaluating patient risk and guiding treatment protocols, comprising:
acquiring image data of a target soft tissue location within a patient's body while the location is subjected to a given load;
estimating the in-vivo load exerted on the tissue;
using the image data for tracking the deformation of the soft tissue location when subjected to the given load, to determine mechanical properties of the target soft tissue location; and
using the determined mechanical properties to evaluate patient risk for guiding treatment protocols.

14. Methodology according to claim 13, wherein:
the target soft tissue location within a patient's body comprises the aorta; and
tracking the deformation of the aorta includes tracking deformation of the aorta during and after associated ventricular ejection.

15. Methodology according to claim 13, wherein:
the target soft tissue location within a patient's body comprises the heart; and
the methodology further comprises determining left ventricle (LV) chamber stiffness of the heart, for providing a biomechanical marker of the rate and extent of LV remodeling.

16. Methodology according to claim 15, wherein determining left ventricle (LV) chamber stiffness of the heart comprises determining at least one of increases in LV wall thickness or LV myocardial stiffness.
